SEQUENCE LISTING (15 sequences)

SEQ ID NO: 1 - protein, 56 aa, Staphylococcus aureus
AQHDEAQQNAFYQVLNMPNLNADQRNGFIQSLKDDPSQSANVLGEAQKLNDSQAPK

SEQ ID NO: 2 - protein, 61 aa, Staphylococcus aureus
ADAQQNKFNKDQQSAFYEILNMPNLNEEQRNGFIQSLKDDPSQSTNVLGEAKKLNESQAPK

SEQ ID NO: 3 - protein, 58 aa, Staphylococcus aureus
ADNNFNKEQQNAFYEILNMPNLNEEQRNGFIQSLKDDPSQSANLLAEAKKLNESQAPK

SEQ ID NO: 4 - protein, 58 aa, Staphylococcus aureus [sequence omitted]

SEQ ID NO: 5 - protein, 58 aa, Staphylococcus aureus
ADNKFNKEQQNAFYEILHLPNLTEEQRNGFIQSLKDDPSVSKEILAEAKKLNDAQAPK

SEQ ID NO: 6 - protein, 290 aa, Artificial Sequence
  Synthetic peptide derived from C domain of Protein A of Staphylococcus aureus (C-(5d-58K))
ADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPK

SEQ ID NO: 7 - protein, 290 aa, Artificial Sequence
  Synthetic peptide derived from C domain of Protein A of Staphylococcus aureus (C-(1d-7K))
ADNRFNKEQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPR

SEQ ID NO: 8 - protein, 290 aa, Artificial Sequence
  Synthetic peptide derived from C domain of Protein A of Staphylococcus aureus (C-K04R/K07R/G29A/S33R /K35R/K42R/K49Q/K50R/K58R.5d)
ADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPR

SEQ ID NO: 9 - DNA, 881 bp, Artificial Sequence
  Synthetic DNA derived from C domain of Protein A of Staphylococcus aureus (C-(NonK))
ctgcagataaccgttttaaccgtgaacaacaaaacgctttctacgaaatcctgcacttgccaaaccttactgaagaacaacgtaatgctttcatccaacgtctgcgcgatgatccatctgtatcccgtgaaattttggcagaggctcaacgacttaacgacgctcaggcgcctcgcgctgataaccgcttcaaccgagaacaacaaaacgctttttatgaaatccttcacctgccaaatcttacagaagaacaacgcaacgcattcattcaacgcttgcgtgatgacccttccgttagccgagagatcctggctgaagcacaacgtttgaatgatgcgcaagcaccacgtgctgataatcgattcaaccgtgaacaacaaaatgcattctacgaaatcttgcaccttcctaacctgactgaagagcagcgtaacgcttttatccagcgtttgagagacgatccatctgtctcccgtgaaattctcgcagaagcgcaacgcctgaacgatgctcaagctccgcgtgcagacaaccgtttcaatcgcgaacagcaaaacgcgttttatgaaattctgcatcttccaaacttgacagaggaacaacgcaatgctttcatccaacgactgcgtgatgatccgagcgtttctcgagaaatcttggctgaagcacaacgtctgaacgacgctcaagctccacgagcggataacagatttaaccgtgaacaacaaaatgctttctacgagatcttgcaccttccgaacctgactgaagaacaacgtaacgcatttattcagagattgcgcgatgacccatccgtaagccgtgagatcctggcagaagctcaacgtttgaatgatgcacaagctccacgttaatctaga

SEQ ID NO: 10 - DNA, 33 bp, Artificial Sequence
  primer 1
ttcgctgcagataaccgttttaaccgtgaacaa

SEQ ID NO: 11 - DNA, 30 bp, Artificial Sequence
  primer 2
actatctagattattttggagcttgtgcat

SEQ ID NO: 12 - DNA, 33 bp, Artificial Sequence
  primer 3
ttcgctgcagataaccgttttaacaaagaacaa

SEQ ID NO: 13 - DNA, 30 bp, Artificial Sequence
  primer 4
actatctagattaacgtggagcttgtgcat

SEQ ID NO: 14 - DNA, 33 bp, Artificial Sequence
  primer 5
ttcgctgcagataacaaatttaaccgtgaacaa

SEQ ID NO: 15 - protein, 290 aa, Artificial Sequence
  Synthetic peptide derived from C domain of Protein A of Staphylococcus aureus (C-(1d-4K))
ADNKFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPRADNRFNREQQNAFYEILHLPNLTEEQRNAFIQRLRDDPSVSREILAEAQRLNDAQAPR

(19) United States
(12) Patent Application Publication
(10) Pub. No.: US 2018/0215836 A1
(43) Pub. Date: Aug. 2, 2018

(54) IMMUNOGLOBULIN-BINDING MODIFIED PROTEIN

(71) Applicant: Kaneka Corporation, Osaka (JP)

(72) Inventors: Masakatsu Nishihachijyo, Hyogo (JP); Fuminori Konoike, Hyogo (JP); Yoshiyuki Nakano, Hyogo (JP); Masayuki Takano, Hyogo (JP); Keita Yamashita, Osaka (JP)

(73) Assignee: Kaneka Corporation, Osaka (JP)

(21) Appl. No.: 15/883,569

(22) Filed: Jan. 30, 2018

Related U.S. Application Data
(63) Continuation of application No. PCT/JP2016/072356, filed on Jul. 29, 2016.

(30) Foreign Application Priority Data
Jul. 31, 2015 (JP) 2015-152245

(51) Int. Cl. C07K 17/02; C07K 1/22; B01D 15/38; C07K 16/06; B01J 20/32

(52) U.S. Cl. CPC C07K 17/02; C07K 1/22; B01D 15/3809; C07K 2317/92; B01J 20/3274; C07K 2317/52; C07K 16/065

(57) ABSTRACT

A protein includes two or more amino acid sequences, wherein each amino acid sequence is derived from a sequence selected from the group consisting of SEQ ID NOs: 1 to 5. The amino acid sequence closest to the N-terminus includes more Lys residues than the other amino acid sequence(s), and in the amino acid sequence closest to the N-terminus, a total number of Lys in positions 1 to 38 is equal to or greater than a total number of Lys in position 39 and subsequent positions.

### TECHNICAL FIELD

One or more embodiments of the present invention relate to a protein specifically binding to a target substance, a ligand affinity separation matrix on which the protein is immobilized, and a separation purification method using the matrix.

### BACKGROUND

One of important functions of proteins is to specifically bind to a particular molecule. This function plays a key role in immunoreactions or signal transduction in vivo. Meanwhile, techniques utilizing this function for separation and purification of useful substances have also been actively developed. An example of an actual industrial application is Protein A affinity separation matrices that are used to purify (capture) antibody drugs from animal cell cultures at one time at high purity levels.

The antibody drugs developed so far are generally monoclonal antibodies, which are massively produced by, for example, recombinant cell culture techniques. The “monoclonal antibodies” refer to antibodies that are produced by clones of a single antibody-producing cell. Almost all antibody drugs currently available on the market are classified into immunoglobulin G (IgG) subclasses based on their molecular structure. Protein A is a cell wall protein produced by the gram-positive bacterium Staphylococcus aureus and contains a signal sequence S, five immunoglobulin-binding domains (E domain, D domain, A domain, B domain, and C domain) and a cell wall-anchoring domain known as XM region (Non-Patent Literature 1). The initial purification step (capture step) in antibody drug production processes usually employs an affinity chromatography column where Protein A is immobilized as a ligand on a water-insoluble carrier (Non-Patent Literatures 1, 2, and 3).

Various techniques for improving the performance of Protein A columns have been developed. Technical developments have also been made in ligands. Initially, wild-type Protein A has been used as a ligand, but recombinant Protein A altered by protein engineering has also appeared as a ligand in many techniques for improving the column performance. In particular, some of the Protein A engineering techniques developed focus on how to immobilize Protein A ligands onto water-insoluble carriers.

A Protein A variant obtained by introducing a mutation of one cysteine residue (Cys) into Protein A is site-specifically immobilized onto a carrier via Cys (Patent Literature 1). A Protein A variant in which the ratio between the numbers of lysine residues (Lys) on the antibody binding surface and the non-binding surface of Protein A has been changed is immobilized onto a carrier at multiple sites while moderately controlling the orientation of the ligand during the immobilization (Patent Literature 2). Moreover, Protein A variants having an amino acid sequence from which Lys or Cys has been completely deleted are immobilized onto a carrier via their N-terminus (α-amino group) or C-terminus (special tag) (Patent Literatures 3 to 8). Thus, technical developments in immobilizing protein ligands onto affinity separation matrices have revolved around Protein A columns which require high performance because of their high industrial usefulness.

### CITATION LIST

### Patent Literature

Patent Literature 1: JP 2007-252368 A
Patent Literature 2: WO 1997/017361
Patent Literature 3: JP 2008-115151 A
Patent Literature 4: JP 2008-266219 A
Patent Literature 5: WO 2012/133342
Patent Literature 6: WO 2012/133349
Patent Literature 7: WO 2014/046278
Patent Literature 8: WO 2015/034000

### Non Patent Literature

Non-Patent Literature 1: Hober S. et al., “J. Chromatogr. B”, 2007, 848, pp. 40-47
Non-Patent Literature 2: Low D. et al., “J. Chromatogr. B”, 2007, 848, pp. 48-63
Non-Patent Literature 3: Roque A. C. A. et al., “J. Chromatogr. A”, 2007, 1160, pp. 44-55

### SUMMARY

One or more embodiments of the present invention provide an immunoglobulin-binding engineered protein to allow us to prepare an affinity separation matrix having high antibody binding capacity.

The inventors have designed a numerous recombinant variants of Protein A, obtained the variants by protein engineering and genetic engineering techniques, and evaluated the culture productivity of the variants. As a result, the inventors have found that the antibody binding capacity of an affinity separation matrix in which a protein is immobilized as a ligand can be improved when the protein is a protein having two or more amino acid sequences derived from a Protein A domain, wherein the domain-derived amino acid sequence closest to the N-terminus contains a larger number of Lys residues than the other domain-derived amino acid sequence(s), wherein in the domain-derived amino acid sequence closest to the N-terminus, the number of Lys residues within position 39 and subsequent positions does not exceed the number of Lys residues within positions 1 to 38.

One or more embodiments of the present invention relate to a protein, having two or more amino acid sequences derived from any of the E, D, A, B, and C domains of Protein A of SEQ ID NOs: 1 to 5, wherein the domain-derived amino acid sequence closest to the N-terminus contains a larger number of Lys residues than the other domain-derived amino acid sequence(s), wherein in the domain-derived amino acid sequence closest to the N-terminus, the number of Lys residues within position 39 and subsequent positions does not exceed the number of Lys residues within positions 1 to 38.

In one or more embodiments, in the domain-derived amino acid sequence closest to the N-terminus, Lys is present only within positions 1 to 8 and/or positions 51 to 58, and the number of Lys residues within positions 51 to 58 does not exceed the number of Lys residues within positions 1 to 8.

In one or more embodiments, Lys is present only within positions 1 to 8 of the domain-derived amino acid sequence closest to the N-terminus and at position 58 of each domain.

In one or more embodiments, the other domain-derived amino acid sequence(s) contains no Lys.

In one or more embodiments, Lys is present only within positions 1 to 8 of the domain-derived amino acid sequence closest to the N-terminus.

In one or more embodiments, Lys is present only at position 4 and/or position 7 of the domain-derived amino acid sequence closest to the N-terminus.

In one or more embodiments, at least 90% of the following amino acid residues are retained: Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57, wherein the residue numbers indicated are for the C domain.

One or more embodiments of the present invention further relate to a DNA, encoding the protein.

One or more embodiments of the present invention further relate to a vector, containing the DNA.

One or more embodiments of the present invention further relate to a transformant, produced by transforming a host cell with the vector.

One or more embodiments of the present invention further relate to a method for producing the protein, the method including using the transformant, or a cell-free protein synthesis system including the DNA.

One or more embodiments of the present invention further relate to an affinity separation matrix, including the protein as an affinity ligand immobilized on a carrier made of a water-insoluble base material.

In one or more embodiments, the affinity separation matrix binds to a protein containing an immunoglobulin Fc region.

In one or more embodiments, the protein containing an immunoglobulin Fc region is an immunoglobulin G or an immunoglobulin G derivative.

One or more embodiments of the present invention further relate to a method for preparing the affinity separation matrix, the method including immobilizing the protein as an affinity ligand onto a carrier made of a water-insoluble base material.

One or more embodiments of the present invention further relate to a method for purifying a protein containing an immunoglobulin Fc region, the method including adsorbing a polypeptide containing an immunoglobulin Fc region onto the affinity separation matrix.

One or more embodiments of the protein of the present invention allow us to prepare an affinity separation matrix having high antibody binding capacity.

### BRIEF DESCRIPTION OF DRAWINGS

FIG. 1 shows a table for comparison of the sequences of the E, D, A, B, and C domains of Protein A from Staphylococcus sp.

### DETAILED DESCRIPTION OF EMBODIMENTS

The protein according to one or more embodiments of the present invention has two or more amino acid sequences derived from any of the E, D, A, B, and C domains of Protein A of SEQ ID NOs: 1 to 5, wherein the domain-derived amino acid sequence closest to the N-terminus contains a larger number of Lys residues than the other domain-derived amino acid sequence(s), wherein in the domain-derived amino acid sequence closest to the N-terminus, the number of Lys residues within position 39 and subsequent positions does not exceed the number of Lys residues within positions 1 to 38

Substitutions of amino acid residues are denoted herein with the code for the wild-type or non-mutated type amino acid residue, followed by the position number of the substitution, followed by the code for changed amino acid residue. For example, a substitution of Ala for Gly at position 29 is represented by G29A.

The term “protein” is intended to include any molecule having a polypeptide structure and also encompass fragmented polypeptide chains and polypeptide chains linked by peptide bonds.

In general, the term “domain” refers to a higher-order protein structural unit having a sequence that consists of several tens to hundreds of amino acids, enough to fulfill a certain physicochemical or biochemical function. The domain as used herein particularly refers to a domain that binds to a protein containing an immunoglobulin Fc region.

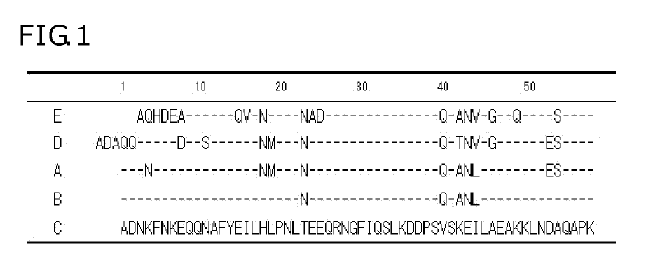

The domain-derived amino acid sequence refers to an amino acid sequence before the amino acid substitution. The domain-derived amino acid sequence is not limited only to the wild-type amino acid sequences of the E, D, A, B, and C domains of Protein A, and may include any amino acid sequence partially engineered by amino acid substitution, insertion, deletion, or chemical modification, provided that it forms a protein having the ability to bind to an Fc region. Examples of the domain-derived amino acid sequence include the amino acid sequences of the E, D, A, B, and C domains of Staphylococcus Protein A of SEQ ID NOs: 1 to 5. Examples also include proteins having amino acid sequences obtained by introducing a substitution of Ala for Gly corresponding to position 29 of the C domain into the E, D, A, B, and C domains of Protein A. In addition, the Z domain produced by introducing A1V and G29A mutations into the B domain corresponds to the domain-derived amino acid sequence because it also has the ability to bind to an Fc region. The domain-derived amino acid sequence may be a domain having high chemical stability or a variant thereof. These domains can be aligned as shown in FIG. 1. For example, the residue corresponding to position 31 of the C domain corresponds to that at the same position 31 of the A or B domain, at position 29 of the E domain, and at position 34 of the D domain.

The amino acid sequence derived from any of the domains may be an amino acid sequence that meets at least one of the following conditions (1) to (4):

(1) the amino acid residue in the corresponding domain that corresponds to position 29 of the C domain is Ala, Val, Leu, Ile, Phe, Tyr, Trp, Thr, Ser, Asp, Glu, Arg, His, or Met;
(2) the amino acid residue in the corresponding domain that corresponds to position 33 of the C domain is Leu, Ile, Phe, Tyr, Trp, Thr, Asp, Glu, Asn, Gln, Arg, His, or Met;
(3) the amino acid residue in the corresponding domain that corresponds to position 36 of the C domain is Leu, Ile, Phe, Tyr, Trp, Glu, Arg, His, or Met; and
(4) the amino acid residue in the corresponding domain that corresponds to position 37 of the C domain is Leu, Ile, Phe, Tyr, Trp, Glu, Arg, His, or Met.

The amino acid sequence derived from any of the domains may be an amino acid sequence that meets at least one of the following conditions (1) to (4):

(1) the amino acid residue in the corresponding domain that corresponds to position 29 of the C domain is Ala, Glu, or Arg;
(2) the amino acid residue in the corresponding domain that corresponds to position 33 of the C domain is Leu, Thr, Glu, Gln, Arg, or His;
(3) the amino acid residue in the corresponding domain that corresponds to position 36 of the C domain is Ile or Arg; and
(4) the amino acid residue in the corresponding domain that corresponds to position 37 of the C domain is Leu, Ile, Glu, Arg, or His.

The domain-derived amino acid sequence may have at least 85%, at least 90%, or at least 95% sequence identity to any of the E, D, A, B, and C domains of Protein A of SEQ ID Nos:1 to 5.

The protein according to one or more embodiments of the present invention has two or more amino acid sequences derived from any of the E, D, A, B, and C domains of Protein A of SEQ ID Nos:1 to 5. The number of domains in the protein according to one or more embodiments of the present invention may be two or more, three or more, four or more, five or more, or six or more. The number of domains in the protein according to one or more embodiments of the present invention may be 20 or less, 10 or less, eight or less, or six or less.

In one or more embodiments of the present invention, the amino acid sequence of the domain closest to the N-terminus may be different from that of the other domain(s). In the case where a plurality of domains are connected to the C-terminal end of the domain closest to the N-terminus, the domains other than the domain closest to the N-terminus may form a protein that includes a homopolymer (e.g. homodimer, homotrimer) consisting of linked single immunoglobulin-binding domains, terminated with Lys, or a protein that includes a heteropolymer (e.g. heterodimer, heterotrimer) consisting of linked different immunoglobulin-binding domains, terminated with Lys.

The monomeric proteins may be linked to each other by, for example, but not limited to: a method that does not use an amino acid residue as a linker; or a method that uses one or more amino acid residues. The number of amino acid residues used as the linker is not particularly limited, and may be such that the three-dimensional conformation of the monomeric proteins does not become unstable.

The term “linker” herein means a linker between domains, and refers to a linking portion of linked monomeric proteins (single domains), i.e., a region between the C-terminal region of a domain sequence closer to the N-terminus and the N-terminal region of a domain sequence closer to the C-terminus. In the case of a protein including N number of domains linked in tandem, the number of linkers in the protein is N−1. In other words, the “linker” herein refers to a region that consists of at least two amino acid residues, including the C-terminal amino acid of a domain closer to the N-terminus and the N-terminal amino acid of a domain closer to the C-terminus, which are connected to each other.

The linker may be an N-/C-terminal sequence of a domain that does not assume a specific secondary structure or that is located on the border between domains. In this case, the linker on the N-terminal side of an immunoglobulin G-binding domain of Protein A, for example, includes amino acid residues corresponding to positions 1 to 6, positions 1 to 5, positions 1 to 4, positions 1 to 3, or positions 1 to 2, of the C domain, and contains at least the N-terminal amino acid residue. It should be noted that although the E and D domains are different from the C domain in full length, their amino acid residues corresponding to the above-mentioned amino acids of the C domain correspond to the linker. Likewise, the linker on the C-terminal side of an immunoglobulin G-binding domain of Protein A, for example, includes amino acid residues corresponding to positions 55 to 58, positions 56 to 58, or positions 57 to 58, of the C domain, and contains at least the C-terminal amino acid residue at position 58.

The protein according to one or more embodiments of the present invention having two or more domains is characterized in that the domain-derived amino acid sequence closest to the N-terminus contains a larger number of Lys residues than the other domain-derived amino acid sequence(s). For example, in the case where the protein according to one or more embodiments of the present invention consists of five domains and the domain-derived amino acid sequence closest to the N-terminus contains two Lys residues, the number of Lys residues in each of the other four domain-derived amino acid sequences is less than two. In order to avoid formation of immobilization sites when the protein is immobilized as an affinity ligand onto a carrier, the Lys contained in the other domain-derived amino acid sequences may be not exposed on the protein surface.

The number of Lys residues in the domain-derived amino acid sequence closest to the N-terminus may be larger by at least one than the number of Lys residues in each of the other domain-derived amino acid sequences. The number of Lys residues in the domain-derived amino acid sequence closest to the N-terminus may also be six, five, four, three, two, or one.

The protein according to one or more embodiments of the present invention having two or more domains is also characterized in that in the domain-derived amino acid sequence closest to the N-terminus, the number of Lys residues within position 39 and subsequent positions does not exceed the number of Lys residues within positions 1 to 38. Here, the amino acid sequence between position 39 and subsequent positions refers to the amino acid sequence between position 39 and subsequent positions of the C domain of Protein A, or an amino acid sequence of the E, D, A, or B domain of Protein A which is in the same column as the amino acid sequence between position 39 and subsequent positions of the C domain when the E, D, A, B, and C domains are aligned as shown in FIG. 1. The amino acid sequence between positions 1 to 38 refers to the amino acid sequence between positions 1 to 38 of the C domain of Protein A, or an amino acid sequence of the E, D, A, or B domain of Protein A which is in the same column as the amino acid sequence between positions 1 to 38 of the C domain when the E, D, A, B, and C domains are aligned as shown in FIG. 1.

In the domain-derived amino acid sequence closest to the N-terminus, among the two or more domains, the number of Lys residues in the amino acid sequence between position 39 and subsequent positions may be the same as the number of Lys residues in the amino acid sequence between positions 1 to 38, or smaller by at least one than the number of Lys residues in the amino acid sequence between positions 1 to 38. The number of Lys residues in the amino acid sequence between positions 1 to 38 may be three, two, or one.

In the protein having two or more amino acid sequences derived from any of the E, D, A, B, and C domains of Protein A of SEQ ID Nos:1 to 5, as long as the domain-derived amino acid sequence closest to the N-terminus contains a larger number of Lys residues than the other domain-derived amino acid sequences, and in the domain-derived amino acid sequence closest to the N-terminus, the number of Lys residues within position 39 and subsequent positions does not exceed the number of Lys residues within positions 1 to 38, the amino acid sequences are not particularly otherwise limited. They may contain wild-type amino acid residues, non-protein-forming amino acid residues, or non-natural amino acid residues. For production by genetic engineering, wild-type amino acid residues can be suitably used. However, amino acid residues having in a side chain a functional group that is highly reactive in a coupling reaction for immobilization, such as cysteine (Cys) having a thiol group (—SH) in a side chain, are unsuitable as amino acid residues used for substitution. The domain-derived amino acid sequence closest to the N-terminus and the other domain-derived amino acid sequences may contain amino acid substitutions that improve various functions. Examples of such amino acid substitutions include amino acid substitutions which substitute amino acids other than Ala for one or more Gly residues in an amino acid sequence derived from any of the E, D, A, B, and C domains of Protein A, as disclosed in WO 2010/110288. Examples also include amino acid substitutions which introduce at least one amino acid substitution into the amino acid residues at positions 31 to 37 of the A, B, or C domain, amino acid residues at positions 29 to 35 of the E domain, or amino acid residues at positions 34 to 40 of the D domain in an amino acid sequence derived from at least one domain selected from the E, D, A, B, and C domains of Protein A, as disclosed in WO 2011/118699. These amino acid substitutions can reduce affinity for Fab regions to improve antibody elution properties.

In the domain-derived amino acid sequence closest to the N-terminus, Lys may be present only within positions 1 to 8 and/or positions 51 to 58, and the number of Lys residues within positions 51 to 58 does not exceed the number of Lys residues within positions 1 to 8. In the domain-derived amino acid sequence closest to the N-terminus, Lys may be present only within positions 1 to 8 and positions 51 to 58.

The protein according to one or more embodiments of the present invention may contain no Lys in the domain-derived amino acid sequence second closest to the N-terminus or subsequent ones. The protein may contain Lys only within positions 1 to 8 of the domain-derived amino acid sequence closest to the N-terminus and at position 58 of each of the domains including the domain closest to the N-terminus, the domain second closest to the N-terminus, and subsequent domains.

The protein according to one or more embodiments of the present invention may contain Lys only within positions 1 to 8 of the domain-derived amino acid sequence closest to the N-terminus. In this case, Lys may be present only at position 4 and/or position 7 of the domain-derived amino acid sequence closest to the N-terminus.

The number of Lys residues can be controlled by substituting an amino acid residue other than Lys for Lys in the domain-derived amino acid sequence. The amino acid sequence obtained by substituting an amino acid residue other than Lys for Lys may, for example, be the following amino acid sequence: RFX₁X₂EQQNAFYEILHX₃PNLTEEQRNX₄FIQX₅LX₆X₇X₈PSVSREX₉LAEAX₁₀X₁₁LND AQAPX₁₂ wherein X₁=D, E, N, or Q; X₂=E or R; X₃=L, M, or I; X₄=A, E, F, R, Y, or W; X₅=D, E, H, I, L, Q, R, S, T, or V; X₆=H, I, or R; X₇=D, I, or R; X₈=D or E; X₉=I, L, or V; X₁₀=R or Q; X₁₁=H or R; X₁₂=R, G, or K, as disclosed in WO 2012/133342. Moreover, it may be an amino acid sequence derived from any of the E, D, A, B, and C domains of Protein A which contains amino acid substitutions for all lysine residues (Lys) and which is terminated with Lys, as disclosed in WO 2012/133349. Moreover, it may be an amino acid sequence including two or more amino acid sequences derived from any domain selected from the E, D, A, B, and C domains of Protein A which contain amino acid substitutions for all lysine residues (Lys), wherein the amino acid sequences are connected to each other through a linker, and at least one linker contains a lysine residue (Lys) or a cysteine residue (Cys), as disclosed in WO 2014/046278. At least half of the substitutions for Lys may be substitutions with arginine (Arg). This is because Arg is a basic amino acid having similar properties to Lys, and a substitution of Lys with Arg causes a relatively small effect on the properties of the whole protein.

In the protein according to one or more embodiments of the present invention, at least 90%, or at least 95%, of the following 20 amino acid residues are retained: Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57 (the residue numbers indicated are for the C domain).

Moreover, the amino acid sequence of the whole protein may have at least 85%, at least 90%, or at least 95% sequence identity to the amino acid sequence before mutagenesis. The sequence identity of amino acid sequences can be analyzed by a person skilled in the art by direct comparison of the sequences; specifically, it may be analyzed using commercially available sequence analysis software, for example.

The protein according to one or more embodiments of the present invention may be a fusion protein produced by fusion with another protein having a different function. Examples of the fusion protein include, but are not limited to, those fused with albumin or GST (glutathione S-transferase). The protein according to one or more embodiments of the present invention may also be one fused with a nucleic acid (e.g. a DNA aptamer), a drug (e.g. an antibiotic), or a polymer (e.g. polyethylene glycol (PEG)).

One or more embodiments of the present invention also relate to a DNA having a base sequence encoding the protein obtained as above. The DNA may be any DNA having a base sequence that is translated into the amino acid sequence of the protein. Such a DNA can be obtained by common known techniques, such as polymerase chain reaction (hereinafter abbreviated as PCR). Alternatively, it can be synthesized by known chemical synthesis techniques or may be available from DNA libraries. A codon in the base sequence of the DNA may be replaced with a degenerate codon, and the base sequence is not necessarily the same as the original base sequence, provided that the coding base sequence is translated into the same amino acids.

Site-directed mutagenesis for modifying the base sequence of the DNA may be performed by, for example, recombinant DNA technology or PCR as follows.

Specifically, in the case of mutagenesis by recombinant DNA technology, for example, if there are suitable restriction enzyme recognition sequences on both sides of a mutagenesis target site in the gene encoding the protein according to one or more embodiments of the present invention, a cassette mutagenesis method can be used in which these restriction enzyme recognition sequences are cleaved with the restriction enzymes to remove a region containing the mutagenesis target site, and a DNA fragment in which only the target site is mutated by chemical synthesis or other methods is then inserted.

In the case of site-directed mutagenesis by PCR, for example, a double primer method can be used in which PCR is performed using a double-stranded plasmid encoding the protein as a template and two synthetic oligo primers containing complementary mutations in the + and − strands.

The DNA encoding the protein having two or more amino acid sequences derived from any of the E, D, A, B, and C domains of Protein A of SEQ ID Nos:1 to 5 can be obtained by ligating DNAs encoding a single domain. For example, the ligation of DNAs may be accomplished by introducing an appropriate restriction enzyme recognition sequence into a base sequence, fragmenting the sequence with the restriction enzyme, and ligating the double-stranded DNA fragments using a DNA ligase. A single restriction enzyme recognition sequence or a plurality of different restriction enzyme recognition sequences may be introduced.

The method for preparing the DNA encoding the protein having two or more amino acid sequences derived from any of the E, D, A, B, and C domains of Protein A of SEQ ID Nos:1 to 5 is not limited to the methods described above. For example, it may be prepared by applying any of the mutagenesis methods to a DNA encoding Protein A (e.g., see WO 2006/004067). Here, if the base sequences each encoding a monomeric protein in the DNA encoding a polymeric protein are the same, then homologous recombination of the DNA may be induced in transformed host cells. For this reason, the ligated DNAs encoding a monomeric protein may have 90% or lower, or 85% or lower base sequence identity.

The vector according to one or more embodiments of the present invention includes a base sequence encoding the above-described protein or a partial amino acid sequence thereof, and a promoter that is operably linked to the base sequence to function in a host cell. Typically, the vector can be constructed by linking or inserting the above-described gene encoding the protein into an appropriate vector. The vector used for insertion of the gene is not particularly limited, provided that it is capable of autonomous replication in a host cell. The vector may be a plasmid DNA or phage DNA. For example, when Escherichia coli is used as a host cell, examples of the vector include pQE vectors (QIAGEN), pET vectors (Merck), and pGEX vectors (GE Healthcare, Japan). Examples of plasmid vectors useful for the expression of Brevibacillus genes include the known Bacillus subtilis vector pUB110 and pHY500 (JP H02-31682 A), pNY700 (JP H04-278091 A), pNU211R2L5 (JP H07-170984 A), pHT210 (JP H06-133782 A), and the shuttle vector pNCMO2 between Escherichia coli and Brevibacillus (JP 2002-238569 A).

The protein according to one or more embodiments of the present invention can be obtained as a fusion protein with a known protein that serves to assist protein expression or facilitate purification. Examples of such proteins include, but are not limited to, maltose-binding protein (MBP) and glutathione S-transferase (GST). The fusion protein can be produced using a vector that contains the DNA according to one or more embodiments of the present invention and a DNA encoding a protein such as MBP or GST ligated to each other.

In one or more embodiments, the transformant can be produced by introducing the recombinant vector according to one or more embodiments of the present invention into a host cell. The recombinant DNA may be introduced into a host cell by, for example, but not limited to: a calcium ion method, an electroporation method, a spheroplast method, a lithium acetate method, an agrobacterium infection method, a particle gun method, or a polyethylene glycol method. Moreover, in one or more embodiments, the obtained gene function may be expressed in a host cell, for example, by incorporating the gene into a genome (chromosome).

The host cell is not particularly limited, and examples suitable for low-cost mass production are Escherichia coli, Bacillus subtilis, and bacteria (eubacteria) of genera including Brevibacillus, Staphylococcus, Streptococcus, Streptomyces, and Corynebacterium.

The protein according to one or more embodiments of the present invention may be produced by culturing the above-described transformant in a medium to produce and accumulate the protein in the cultured cells (including the periplasmic space thereof) or in the culture medium (extracellularly), and collecting the desired protein from the culture.

Alternatively, the protein according to one or more embodiments of the present invention may be produced by culturing the above-described transformant in a medium to produce and accumulate a fusion protein containing the protein in the cultured cells (including the periplasmic space thereof) or in the culture medium (extracellularly), collecting the fusion protein from the culture, cleaving the fusion protein with an appropriate protease, and collecting the desired protein.

The transformant according to one or more embodiments of the present invention can be cultured in a medium according to common methods for culturing host cells. The medium used to culture the transformant may be any medium that allows for high yield and high efficiency production of the protein. Specifically, carbon and nitrogen sources such as glucose, sucrose, glycerol, polypeptone, meat extracts, yeast extracts, and casamino acids may be used. In addition, the medium may optionally be supplemented with inorganic salts such as potassium salts, sodium salts, phosphates, magnesium salts, manganese salts, zinc salts, or iron salts. When an auxotrophic host cell is used, nutritional substances necessary for its growth may be added. Moreover, antibiotics such as penicillin, erythromycin, chloramphenicol, or neomycin may optionally be added.

Furthermore, a variety of known protease inhibitors, i.e. phenylmethane sulfonyl fluoride (PMSF), benzamidine, 4-(2-aminoethyl)-benzenesulfonyl fluoride (AEBSF), antipain, chymostatin, leupeptin, pepstatin A, phosphoramidon, aprotinin, and ethylenediaminetetraacetic acid (EDTA), and/or other commercially available protease inhibitors may be added at appropriate concentrations in order to reduce the degradation or molecular-size reduction of the target protein caused by host-derived proteases present inside or outside the cells.

Furthermore, in order to ensure accurate folding of the protein according to one or more embodiments of the present invention, molecular chaperones such as GroEL/ES, Hsp70/DnaK, Hsp90, or Hsp104/C1pB may be used (for example, they may be allowed to coexist with the protein by, for example, co-expression or incorporation into a fusion protein). Other methods for ensuring accurate folding of the protein according to one or more embodiments of the present invention may also be used such as, but not limited to, adding an additive for assisting accurate folding to the medium or culturing at low temperatures.

Examples of media that can be used to culture the transformed cell obtained using Escherichia coli as a host include LB medium (1% triptone, 0.5% yeast extract, 1% NaCl) and 2×YT medium (1.6% triptone, 1.0% yeast extract, 0.5% NaCl).

Examples of media that can be used to culture the transformant obtained using Brevibacillus as a host include TM medium (1% peptone, 0.5% meat extract, 0.2% yeast extract, 1% glucose, pH 7.0) and 2SL medium (4% peptone, 0.5% yeast extract, 2% glucose, pH 7.2).

The cell may be aerobically cultured at a temperature of 15° C. to 42° C., or 20° C. to 37° C., for several hours to several days under aeration and stirring conditions to accumulate the protein according to one or more embodiments of the present invention in the cultured cells (including the periplasmic space thereof) or in the culture medium (extracellularly), followed by recovery of the protein. In some cases, the cell may be cultured anaerobically without air.

In the case where the recombinant protein is secreted, the produced recombinant protein can be recovered after the culture by separating the cultured cells from the supernatant containing the secreted protein by a common separation method such as centrifugation or filtration.

Also, in the case where the protein is accumulated in the cultured cells (including the periplasmic space thereof), the protein produced and accumulated in the cells can be recovered, for example, by collecting the cells from the culture medium, e.g. via centrifugation or filtration, followed by disrupting the cells, e.g. via sonication or French press, and/or solubilizing the protein with, for example, a surfactant.

The protein according to one or more embodiments of the present invention can be purified by methods such as affinity chromatography, cation or anion exchange chromatography, and gel filtration chromatography, used alone or in an appropriate combination.

Whether the purified product is the target protein may be confirmed by common methods such as SDS polyacrylamide gel electrophoresis, N-terminal amino acid sequencing, or Western blot analysis.

The protein according to one or more embodiments of the present invention can also be produced using a cell-free protein synthesis system including the DNA. Examples of such cell-free protein synthesis systems include synthesis systems derived from procaryotic cells, plant cells, or higher animal cells.

The protein according to one or more embodiments of the present invention is characterized in that it can be produced by the transformant with improved culture productivity as compared to before the amino acid substitution. The term “culture productivity” can be rephrased as “protein expression level” and refers to the amount per unit volume or per cell of the target recombinant protein produced by culturing the transformant as described above. For example, for secretory production, it may be the concentration of the target protein in the culture supernatant, while in the case where the target recombinant protein is produced in the cells, it can be expressed as the weight of the target protein per cell count or per cell weight. The expression “the protein can be produced by the transformant with improved culture productivity as compared to before the amino acid substitution” means that when a transformant producing a non-mutated protein and a transformant producing the protein containing amino acid substitution are cultured under the same conditions, and then evaluated by the later-described “Method for evaluating culture productivity”, the relative value is higher by at least 5%, for example by at least 10% or by at least 20%.

The culture productivity can be evaluated as follows, for example.

### [Method for Evaluating Culture Productivity]

A transformant producing a non-mutated protein (control) and a transformant producing the protein containing amino acid substitution (evaluation sample) are prepared using the same host and the same transformation method, are cultured under the same conditions. For example, they are aerobically cultured in a medium suited for the host selected from the media mentioned above at an appropriate culture temperature (e.g. 20° C. to 37° C.) under aeration and stirring conditions for several hours to several days to accumulate the protein according to one or more embodiments of the present invention in the cultured cells (including the periplasmic space thereof) or in the culture medium (extracellular secretion), followed by recovery of the protein. The amount of the protein in the cultured cells or culture medium is quantitated. The quantitation may be carried out by high performance liquid chromatography (HPLC), for example. In HPLC analysis, an appropriate pretreatment is performed on the cultured cells or culture medium. For example, in the case where the protein is accumulated in the cells, the cells are disrupted with a device such as a sonicator and then centrifuged to remove the cell residues, followed by filtration. In the case where the protein is extracellularly secreted, the cells are separated from the culture medium by centrifugation, followed by filtration. The control, the evaluation sample, and a reference (a highly purified protein having a predetermined concentration) are analyzed by HPLC, and the concentration of the reference and the analytical data (chromatographic areas) are used to calculate the concentration of the protein in the control or evaluation sample, which is then used to calculate the relative value using the following (Equation 1). The culture productivity is considered improved if the relative value of the evaluation sample may be higher by at least 5%, by at least 10%, or by at least 20% than that of the control.

Relative value (%)=[protein concentration of control]/[protein concentration of evaluation sample] (Equation 1)

The protein according to one or more embodiments of the present invention can be used as an affinity ligand having affinity for an immunoglobulin. An affinity separation matrix can be prepared by a method including immobilizing the protein according to one or more embodiments of the present invention as an affinity ligand onto a carrier made of a water-insoluble base material.

The term “affinity ligand” means a substance (functional group) that selectively captures (binds to) a target molecule from a mixture of molecules by virtue of a specific affinity between the molecules such as antigen-antibody binding, and refers herein to a protein that specifically binds to an immunoglobulin. The term “ligand” as used alone herein is synonymous with “affinity ligand”.

The “immunoglobulin binding properties” can be tested using, for example, but not limited to, biosensors such as Biacore system (GE Healthcare, Japan) based on the surface plasmon resonance principle. The measurement may be carried out under any conditions that allow detection of a binding signal corresponding to the binding of Protein A to an immunoglobulin Fc region. The properties can be easily evaluated at a (constant) temperature of 20° C. to 40° C. and a neutral pH of 6 to 8.

Examples of binding parameters that can be used include affinity constant (KA) and dissociation constant (KD) (Nagata et al., “Real-time analysis of biomolecular interactions”, Springer-Verlag Tokyo, 1998, page 41). In one or more embodiments, the affinity constant of the protein for Fc may be determined in an experimental system using Biacore system in which human IgG is immobilized on a sensor chip, and each domain variant is added to a flow channel at a temperature of 25° C. and a pH of 7.4. The protein according to one or more embodiments of the present invention may suitably be a protein having an affinity constant (KA) for human IgG of at least 1×10⁵ (M⁻¹), at least 1×10⁶ (M⁻¹), or 1×10⁷ (M⁻¹).

In order to provide an affinity ligand that has high chemical stability under alkali conditions, the amino acid sequence before mutagenesis may be an amino acid sequence derived from the C domain of SEQ ID No:5. Moreover, the amino acid sequence before mutagenesis may be an amino acid sequence derived from the C domain of SEQ ID No:5 in which the amino acid residue corresponding to position 29 is any amino acid residue selected from Ala, Arg, Glu, Ile, Leu, Met, Phe, Trp, and Tyr. Furthermore, at least half of the amino acid residues introduced to all Lys residues may be substitutions with Arg, or all of them may be substitutions with Arg. In the case where all of them are substitutions with Arg, the chemical stability under alkali conditions can be improved as compared to before the mutagenesis.

The chemical stability under alkali conditions may be determined using binding activity to an immunoglobulin or material stability of a polypeptide itself as an index. In the case where material stability of a polypeptide itself is used as an index, the chemical stability under alkali conditions can be evaluated, for example, by electrophoresis in which electrophoresis bands of the polypeptide before and after an alkali treatment are compared. Specifically, comparison of chemical stability may be made by performing standard SDS-PAGE to analyze band intensity via densitometry. If chemical stability is indicated based on the band intensities analyzed via densitometry, the polypeptide according to one or more embodiments of the present invention, after being left in a 0.5 M sodium hydroxide aqueous solution at 25° C. for 24 hours, may have a band intensity of 50% or higher, 60% or higher, 70% or higher, or 80% or higher, of that before the treatment.

Examples of the carrier made of a water-insoluble base material used in one or more embodiments of the present invention include inorganic carriers such as glass beads and silica gel; organic carriers such as synthetic polymers (e.g. cross-linked polyvinyl alcohol, cross-linked polyacrylate, cross-linked polyacrylamide, cross-linked polystyrene) and polysaccharides (e.g. crystalline cellulose, cross-linked cellulose, cross-linked agarose, cross-linked dextran); and composite carriers formed by combining these carriers, such as organic-organic or organic-inorganic composite carriers. Examples of commercially available products include GCL2000 (porous cellulose gel), Sephacryl S-1000 (prepared by covalently cross-linking allyl dextran with methylene bisacrylamide), Toyopearl (methacrylate carrier), Sepharose CL4B (cross-linked agarose carrier), and Cellufine (cross-linked cellulose carrier), although the water-insoluble carrier used in one or more embodiments of the present invention is not limited to the carriers listed above.

In view of the purpose and method of using the affinity separation matrix, the water-insoluble carrier used in one or more embodiments of the present invention should have a large surface area and may be a porous material having a large number of fine pores of an appropriate size. The carrier may be in any form such as bead, monolith, fiber, film (including hollow fiber) or other optional forms.

The ligand may be immobilized by any method that allows the ligand to be covalently bonded to the carrier via the ε-amino group of lysine on the ligand by a conventional coupling process. Moreover, even if some ligands turn out to be immobilized on the carrier via the α-amino group at the N-terminus, the effect according to one or more embodiments of the present invention will not deteriorate because they are immobilized at the protein terminus. The coupling process may be carried out by reacting the carrier with cyanogen bromide, epichlorohydrin, diglycidyl ether, tosyl chloride, tresyl chloride, hydrazine, sodium periodate, or the like to activate the carrier (or introduce a reactive functional group into the carrier surface), and performing a coupling reaction between the carrier and the compound to be immobilized as a ligand to immobilize the ligand; or by adding a condensation reagent such as carbodiimide or a reagent having a plurality of functional groups in the molecule such as glutaraldehyde to a system containing the carrier and the compound to be immobilized as a ligand, followed by condensation and cross-linking for immobilization. Moreover, a spacer molecule consisting of a plurality of atoms may be introduced between the ligand and the carrier, or alternatively, the ligand may be directly immobilized onto the carrier.

The affinity separation matrix according to one or more embodiments of the present invention can be used to separate and purify a protein containing an immunoglobulin Fc region by affinity column chromatography purification techniques. As described earlier, the regions to which immunoglobulin-binding domains bind are broadly specified as Fab regions (particularly Fv regions) and Fc regions. However, since the conformation of antibodies is already known, it is possible to further alter (e.g. fragmentize) the Fab or Fc regions while maintaining the conformation of the regions to which Protein A binds by protein engineering techniques. Accordingly, the present invention is not limited to immunoglobulin molecules containing Fab and Fc regions sufficiently, and derivatives thereof. Therefore, the term “protein containing an immunoglobulin Fc region” refers to a protein containing an Fc region portion to which Protein A binds, and it does not have to contain the complete Fc region as long as Protein A is able to bind to the protein.

Typical examples of the protein containing an immunoglobulin Fc region include, but are not limited to, immunoglobulin G and immunoglobulin G derivatives. The term “immunoglobulin G derivative” is a generic term for engineered artificial proteins to which Protein A can bind, such as chimeric immunoglobulin G in which the domains of human immunoglobulin G are partially replaced and fused with immunoglobulin G domains of another biological species, humanized immunoglobulin G in which complementarity determining regions (CDRs) of human immunoglobulin G are replaced and fused with antibody CDRs of another biological species, immunoglobulin G whose Fc region has a molecularly altered sugar chain, and artificial immunoglobulin G in which the Fv and Fc regions of human immunoglobulin G are fused.

These proteins containing an immunoglobulin Fc region can be purified according to affinity column chromatographic purification techniques using existing commercial Protein A columns (Non-Patent Literature 3). Specifically, a buffer containing the protein containing an immunoglobulin Fc region is adjusted to be neutral, and the resulting solution is passed through an affinity column filled with the affinity separation matrix according to one or more embodiments of the present invention to adsorb the protein containing an immunoglobulin Fc region. Next, an appropriate volume of pure buffer is passed through the affinity column to wash the inside of the column. At this point, the desired protein containing an immunoglobulin Fc region remains adsorbed on the affinity separation matrix according to one or more embodiments of the present invention in the column. Subsequently, an acidic buffer (which may contain a substance for promoting dissociation from the matrix) adjusted to an appropriate pH is passed through the column to elute the desired protein containing an immunoglobulin Fc region, whereby high-level purification can be achieved.

The affinity separation matrix according to one or more embodiments of the present invention can be reused by passing therethrough a pure buffer having an appropriate strong acidity or strong alkalinity which does not completely impair the functions of the ligand compound and the carrier base material (or optionally a solution containing an appropriate modifying agent or an organic solvent) for washing.

One or more embodiments of the present invention further relate to a protein obtained by a separation method using the affinity separation matrix. The protein may be a protein containing an immunoglobulin Fc region. The protein obtained by using the affinity separation matrix is obtained as a high-purity, high-concentration solution, and has properties that maintain its inherent activity such as ability to bind to an antigen, without impairing it.

### EXAMPLES

The following description is offered to illustrate one or more embodiments of the present invention in greater detail by reference to examples, but the scope of the present invention is not limited to these examples. In the examples, operations such as recombinant DNA production and engineering were performed in accordance with the following textbooks, unless otherwise noted: (1) T. Maniatis, E. F. Fritsch, J. Sambrook, “Molecular Cloning/A Laboratory Manual”, 2nd edition (1989), Cold Spring Harbor Laboratory (USA); (2) Masami Muramatsu, “Lab Manual for Genetic Engineering”, 3rd edition (1996), Maruzen Co., Ltd. The materials such as reagents and restriction enzymes used in the examples were commercially available products, unless otherwise specified.

Proteins obtained in the examples are represented by “an alphabetical letter identifying the domain—an introduced mutation (wild for the wild type)”. For example, the wild-type C domain of Protein A is represented by “C-wild”, and a C domain variant containing G29E mutation is represented by “C-G29E”. Variants containing two mutations together are represented by indicating both with a slash. For example, a C domain variant containing G29E and S13L mutations is represented by “C-G29E/S13L”. Proteins consisting of a plurality of single domains linked together are represented by adding a period (.) followed by the number of linked domains followed by “d”. For example, a protein consisting of five linked C domain variants containing G29E and S13L mutations is represented by “C-G29E/S13L.5d”. In the case of proteins containing a mutation in the domain second closest to the N-terminus and subsequent domains, and a different mutation in the domain closest to the N-terminus, the mutations in the second and subsequent domains are represented as described above, and the mutation in the domain closest the N-terminus is added after the number of linked domains. For example, a protein consisting of five linked C domain variants containing K04Q mutation in the domain closest to the N-terminus and K04R mutation in the second to fifth domains is represented by “C-KO4R.5d-K04Q”.

### (Example 1) Preparation of C Domain Variant

Expression plasmids of two C domain variants of Protein A containing only one Lys residue in the sequence, C-(5d-58K) (SEQ ID No:6), C-(1d-7K) (SEQ ID No:7), and C-(1d-4K) (SEQ ID No:15), were prepared by the procedure described below. The variants have the following basic sequence: C-K04R/K07R/G29A/S33R/K35R/K42R/K49Q/K50R/K58R.5d (SEQ ID No:8; hereinafter, referred to as C-(NonK)). The variant C-(5d-58K) contains Lys at position 58 of the C-terminal domain; C-(1d-7K) contains Lys at position 7 of the N-terminal domain; and C-(1d-4K) contains Lys at position 4 of the N-terminal domain.

The total synthesis of a DNA (SEQ ID No:9) encoding C-(NonK) (SEQ ID No:8) in which PstI and XbaI recognition sites were added to the 5′ and 3′ ends, respectively, was outsourced to Eurofins Genomics K.K. PCR was performed using this synthetic DNA as a template and Primer 1: 5′-TTCGctgcagataacCGTtttaacCGTgaacaa-3′ (SEQ ID No:10) and Primer 2: 5′-ACTATCTAGATTAtttTGGAGCTTGTGCAT-3′ (SEQ ID No:11) to amplify the DNA fragment encoding C-(5d-58K). Similarly, PCR was performed using Primer 3: 5′-TTCGctgcagataacCGTtttaacAAAgaacaa-3′ (SEQ ID No:12) and Primer 4: 5′-ACTATCTAGATTAacgTGGAGCTTGTGCAT-3′ (SEQ ID No:13) to amplify the DNA fragment encoding C-(1d-7K). Similarly, PCR was performed using Primer 5: 5′-TTCGctgcagataacAAAtttaacCGTgaacaa-3′ (SEQ ID No:14) and Primer 4: 5′-ACTATCTAGATTAacgTGGAGCTTGTGCAT-3′ (SEQ ID No:13) to amplify the DNA fragment encoding C-(1d-7K). The obtained DNA fragment was digested with restriction enzymes PstI and XbaI (Takara Bio, Inc.) and ligated to a Brevibacillus expression vector pNCMO2 (Takara Bio, Inc.) digested with the same restriction enzymes to construct an expression plasmid in which a DNA encoding the amino acid sequence of SEQ ID No:6 was inserted into the Brevibacillus expression vector pNCMO2. The plasmid was prepared using Escherichia coli JM109. Plasmids were similarly prepared for DNAs encoding C-(NonK), C-(1d-7K) (SEQ ID No:7), and C-(1d-4K) (SEQ ID No:15).

Brevibacillus choshinensis SP3 (Takara Bio, Inc.) was transformed with the obtained plasmids, and the recombinant strains secreting C-(NonK), C-(5d-58K), or C-(1d-7K) were grown. Each recombinant strain was cultured with shaking at 30° C. for three days in 30 mL of A medium (3.0% polypeptone, 0.5% yeast extract, 3% glucose, 0.01% magnesium sulfate, 0.001% iron sulfate, 0.001% manganese chloride, 0.0001% zinc chloride) containing 60 μg/mL neomycin.

After the culture, the culture medium was sampled to analyze turbidity at 600 nm using a spectrophotometer. The cells were removed from the culture medium by centrifugation (15,000 rpm, 25° C., five minutes) to measure the concentration of the C domain variant in the culture supernatant by high performance liquid chromatography. Next, acetic acid was added to the culture supernatant to adjust the pH to 4.5, followed by standing for one hour to precipitate the target protein. The precipitate was recovered by centrifugation and dissolved in a buffer (50 mM Tris-HCl, pH 8.5). Next, the target protein was purified by anion exchange chromatography using HiTrap Q column (GE Healthcare Bio-Sciences). Specifically, the target protein solution was applied to HiTrap Q column equilibrated with anion exchange buffer A (50 mM Tris-HCl, pH 8.0), and washed with anion exchange buffer A, followed by elution with a salt gradient using anion exchange buffer A and anion exchange buffer B (50 mM Tris-HCl, 1 M NaCl, pH 8.0) to separate the target protein eluted in the middle of the gradient. The separated target protein solution was dialyzed with ultrapure water, and the dialyzed aqueous solution was used as a finally purified sample. All processes of protein purification by column chromatography were carried out using AKTA avant system (GE Healthcare Bio-Sciences).

### (Example 2) Evaluation of C Domain Variant-Immobilized Affinity Separation Matrix

A crystalline cross-linked cellulose (available from JNC, a gel prepared as described in JP 2009-242770 A and US 2009/0062118) was used as a water-insoluble base material. This base material (3.5 mL) was put on a glass filter (17G-2 available from TOP) and substituted with 0.01 M citrate buffer, pH 3 (prepared using trisodium citrate dihydrate (Wako Pure Chemical Industries, Ltd.), citric acid monohydrate, and RO water). Then, the liquid volume was adjusted to 6 mL in a centrifuge tube (50 mL, Iwaki Glass Co., Ltd.). To this tube was added an aqueous solution prepared by dissolving 22.5 mg of sodium periodate (Wako Pure Chemical Industries, Ltd.) into 2 mL of RO water, and the mixture was shaken at 6° C. for about 30 minutes using a mix rotor (mix rotor MR-3 1-336-05 available from Az One Corporation). The material was washed on a glass filter with an adequate amount of RO water, whereby a formyl group-containing carrier was prepared.

The formyl group-containing carrier (3.5 mL) was substituted on a glass filter with 0.6 M citrate buffer, pH 12 (prepared using trisodium citrate dihydrate (Wako Pure Chemical Industries, Ltd.), sodium hydroxide, and RO water). Then, the total volume was adjusted to 7.5 mL in a centrifuge tube. To this tube was added each of the purified samples prepared in Example 1 and the mixture was shaken at 6° C. for 23 hours using a mix rotor.

Then, a 2.4 M citric acid aqueous solution (prepared using citric acid monohydrate (Wako Pure Chemical Industries, Ltd.) and RO water) was added to adjust the pH to 8, and shaking was continued at 6° C. for four hours. Subsequently, 1.93 mL of a 5.5% by weight dimethylamine borane aqueous solution (prepared using dimethylamine borane (Wako Pure Chemical Industries, Ltd.) and RO water) was added and the mixture was shaken at 25° C. for 18 hours. After the reaction, the reaction solution was measured for maximum absorbance at 275 nm (ABS 276).

The carrier was washed on a glass filter with RO water until the electric conductivity of the washing filtrate reached 5 μS/cm or lower, and further washed sequentially with a 0.1 M citric acid aqueous solution (citric acid monohydrate), a mixed aqueous solution of sodium hydroxide and sodium sulfate (0.05 M sodium hydroxide, 0.5 M sodium sulfate), and a citrate buffer (0.5 M trisodium citrate dihydrate-citric acid monohydrate, pH=6). The carrier was washed finally with RO water until the electric conductivity of the washing filtrate reached 5 μS/cm or lower, whereby affinity separation matrices in which each of the samples prepared in Example 1 was immobilized as a ligand were prepared. The dynamic binding capacity of these affinity separation matrices was measured under the following conditions. The immobilization yields and the dynamic binding capacities (DBC) are shown in Table 1

### <Dynamic Binding Capacity Measurement>

### (1) Preparation of Solution

The following solutions A to E and a neutralization solution were prepared and degassed before use.

Solution A: A PBS buffer having a pH of 7.4 was prepared using phosphate buffered saline (Sigma) and RO water (reverse osmosis purified water).

Solution B: A 35 mM sodium acetate aqueous solution having a pH of 3.5 was prepared using acetic acid, sodium acetate, and RO water.

Solution C: A 1 M acetic acid aqueous solution was prepared using acetic acid and RO water.

Solution D: A 3 mg/mL IgG aqueous solution was prepared using Gammagard (polyclonal antibody, Baxter) and the solution A.

Solution E: An aqueous solution of 0.1 M NaOH and 1 M NaCl was prepared using sodium hydroxide, sodium chloride, and RO water.

Neutralization solution: 2 M Tris(hydroxymethyl)aminomethane was prepared using tris(hydroxymethyl)aminomethane and RO water.

### (2) Filling, Preparation

AKTAexplorer 100 (GE Healthcare) was used as a column chromatography system. A Tricorncolumn (GE Healthcare) having a diameter of 0.5 cm and a height of 15 cm was filled with 3 mL of the affinity separation matrix, followed by passing a 0.2 M NaCl aqueous solution (in RO water) through the column at a linear flow rate of 230 cm/h for 15 minutes. A 15 mL collection tube was attached to the fraction collector, and the neutralization solution was put into the eluate collection tube in advance.

### (3) IgG Purification

The solution A (15 mL) was passed through the column, followed by passing of a necessary amount of the solution D. Subsequently, the solution A (21 mL) and then the solution B (12 mL) were passed therethrough to elute IgG. Thereafter, the solution C (6 mL), the solution E (6 mL), and the solution A (15 mL) were passed therethrough. The flow rate for each solution was 0.5 mL/min or 1 mL/min so that the contact time with the adsorbent was six minutes or three minutes.

### (4) Dynamic Binding Capacity

The dynamic binding capacity of IgG was determined from the amount of IgG adsorbed on the affinity separation matrix before 5% breakthrough of IgG and the volume of the affinity separation matrix.

TABLE 1

| | | | DBC (mg/m L-gel) | |
|---|---|---|---|---|
| | Immobilization | | Contact | Contact |
| | amount | Immobilization | time | time |
| Ligand | (mg/mL-gel) | yield (%) | 3 min | 6 min |
| C-(5d-58K) | 12 | 95% | 42 | 51 |
| C-(1d-4K) | 12 | 95% | 45 | 56 |
| C-(1d-7K) | 12 | 95% | 44 | 55 |

There was not much difference in immobilization yield between C-(5d-58K) immobilized via the C-terminal Lys, C-(1d-7K) immobilized via Lys at position 7 of the N-terminal domain, and C-(1d-4K) immobilized via Lys at position 4 of the N-terminal domain. Meanwhile, the DBC values of the affinity separation matrices of C-(1d-7K) and C-(1d-4K) were higher than that of C-(5d-58K). These results show that immobilization via Lys on the N-terminal side resulted in the preparation of an affinity separation matrix having a higher binding capacity than immobilization via the C-terminal Lys.

### (Example 3) Evaluation of Immobilization Yield of C Domain Variant

An affinity separation matrix was prepared as in Example 2 using the finally purified samples of C-(NonK) and C-(1d-7K) obtained in Example 1, except that the 0.6 M citrate buffer (pH 12) to be mixed with the formyl group-containing carrier was replaced by 0.25 M citrate buffer (pH 12), and the amount of the finally purified sample added was changed.

TABLE 2

| | Immobilization amount | Immobilization |
|---|---|---|
| Ligand | (mg/mL-gel) | yield (%) |
| C-(NonK) | 2 | 16% |
| C-(1d-7K) | 11 | 72% |

The immobilization yield of C-(1d-7K) immobilized via Lys at position 7 of the N-terminal domain was significantly higher than that of C-(NonK) containing no Lys. This shows that immobilization via Lys near the N-terminus of the protein led to a higher immobilization yield than immobilization via the N-terminal α-amino group of the protein.

Although the disclosure has been described with respect to only a limited number of embodiments, those skilled in the art, having benefit of this disclosure, will appreciate that various other embodiments may be devised without departing from the scope of the present invention. Accordingly, the scope of the present invention should be limited only by the attached claims.

What is claimed is:

1. A protein, comprising two or more amino acid sequences,
wherein each amino acid sequence is derived from a sequence selected from the group consisting of SEQ ID NOs: 1 to 5,
wherein the amino acid sequence closest to the N-terminus comprises more Lys residues than the other amino acid sequence(s), and
wherein, in the amino acid sequence closest to the N-terminus, a total number of Lys in positions 1 to 38 is equal to or greater than a total number of Lys in position 39 and subsequent positions.

2. The protein according to claim 1,
wherein, in the amino acid sequence closest to the N-terminus, Lys is present only at one or more positions selected from the group consisting of positions 1 to 8 and 51 to 58, and
wherein a total number of Lys in positions 1 to 8 is equal to or greater than a total number of Lys in positions 51 to 58.

3. The protein according to claim 1,
wherein Lys is present only at one or more positions selected from the group consisting of positions 1 to 8 of the amino acid sequence closest to the N-terminus and position 58 of each amino acid sequence.

4. The protein according to claim 1,
wherein the other amino acid sequence(s) comprises no Lys.

5. The protein according to claim 1,
wherein Lys is present only at one or more positions selected from the group consisting of positions 1 to 8 of the amino acid sequence closest to the N-terminus.

6. The protein according to claim 5,
wherein Lys is present only at one or more positions selected from the group consisting of positions 4 and 7 of the amino acid sequence closest to the N-terminus.

7. The protein according to claim 1,
wherein each amino acid sequence comprises 18 to 20 amino acid residues corresponding to amino acid residues of SEQ ID NO: 5 selected from the group consisting of Gln-9, Gln-10, Phe-13, Tyr-14, Leu-17, Pro-20, Asn-21, Leu-22, Gln-26, Arg-27, Phe-30, Ile-31, Leu-34, Pro-38, Ser-39, Leu-45, Leu-51, Asn-52, Gln-55, and Pro-57.

8. A DNA, encoding the protein according to claim 1.

9. A vector, comprising the DNA according to claim 8.

10. A transformant, produced by transforming a host cell with the vector according to claim 9.

11. A method for producing the protein according to claim 1, the method comprising:
preparing a vector comprising a DNA encoding the protein;
obtaining a transformant by transforming a host cell with the vector; and
producing the protein using the transformant.

12. A method for producing the protein according to claim 1, the method comprising:
preparing a DNA encoding the protein; and
producing the protein using a cell-free protein synthesis system comprising the DNA.

13. An affinity separation matrix, comprising:
a carrier made of a water-insoluble base material; and
an affinity ligand immobilized on the carrier, wherein the affinity ligand is the protein according to claim 1.

14. The affinity separation matrix according to claim 13,
wherein the affinity ligand binds to a protein comprising an immunoglobulin Fc region.

15. The affinity separation matrix according to claim 14,
wherein the protein comprising an immunoglobulin Fc region is an immunoglobulin G or an immunoglobulin G derivative.

16. A method for preparing the affinity separation matrix according to claim 13, the method comprising
immobilizing the protein onto the carrier.

17. A method for purifying a protein comprising an immunoglobulin Fc region, the method comprising
adsorbing a polypeptide comprising an immunoglobulin Fc region onto the affinity separation matrix according to claim 13.
